(19) United States
(12) Patent Application Publication
(10) Pub. No.: US 2014/0170037 A1
(43) Pub. Date: Jun. 19, 2014

(54) DYE ENTRAPPED SOL-GEL FILM BASED TEST STRIP SENSOR FOR NITRITE AND A PROCESS OF PREPARING SAID STRIP SENSOR

(71) Applicants: Santhakumar Dhanya, Trivandrum (IN); Talasila Prasad Rao, Trivandrum (IN)

(72) Inventors: Santhakumar Dhanya, Trivandrum (IN); Talasila Prasad Rao, Trivandrum (IN)

(73) Assignee: COUNCIL OF SCIENTIFIC & INDUSTRIAL RESEARCH, New Delhi (IN)

(21) Appl. No.: 14/233,076

(22) PCT Filed: Aug. 27, 2012
(86) PCT No.: PCT/IB2012/001635

§ 371 Date: Feb. 12, 2014

(30) Foreign Application Priority Data
Aug. 26, 2011 (IN) 2419/DEL/2011

(51) Int. Cl. G01N 33/18

(52) U.S. Cl. CPC G01N 33/182
USPC 422/420; 427/600

(57) ABSTRACT

The present invention relates to test strips that are photo and thermally stable, optically transparent and offer virtually specific absorptiometric sensing of nitrite in natural waters. The process of which is as follows: 1) The entrapment of xanthene dye specifically rhodamine 6G in sol-gel glass. This entrapment is done by delicate balance of coulombic interactions, hydrogen bonding and molecular imprinting effect. 2) In particular, rhodamine 6G entrapped sol-gel layers are formed by first preparing a sol by sonicating a mixture of tetraethoxsilane, water and ethanol for 30 minutes and hydrolysed to form gel by drop wise addition of hydrochloric acid. 3) This gel is further sonicated for 30 minutes after addition of dye and then aged for 15-20 h to obtain dye entrapped sol-gel glass and 4) This is then manually cast as sol-gel films on glass substrates to obtain test strips.

### FIELD OF THE INVENTION

The present invention relates to rhodamine 6G dye entrapped sol-gel film based test strip sensor for nitrite that envisages the entrapping of dye in sol-gel glass but still allows diffusion of smaller analytes into and out of the pores of the sol-gel matrix. More particularly, present invention further relates to process for the preparation of rhodamine 6G dye entrapped sol-gel film which sense nitrite selectively in presence of host of coexisting anionic, cationic and neutral salt species.

### BACKGROUND OF THE INVENTION

Reference may be made to Jeronimo et al [Talanta 72(2007)13] who have reviewed sol-gel film based optical sensors and biosensors upto 2007, Moorcroft et al's review on detection and determination of nitrate and nitrite [Talanta 54 (2001)785] and Dutt and Davis review on strategies of nitrite detection and their application to field analysis [J. Environ.Monit. 4(2002)465].

Another reference may be made to optical sensors [Spectrochim.Acta partA61(2005)1871, Microchem.J.72(2002)193; Eur.Polym.J.45(2009)1516; AsianJ.Chem.17(2005)767; Biosensors and Bioel.17(2002)45]; cellulose acetate polymer film modified optical fibre[Optics. Communications.283(2010)2841]; sol-gel based optic fibre [Sens.ActuatorsB:69(2000)132] and biosensor [Analyst125(2000)1993] reported for nitrite sensing.

Yet another reference may be made to sol-gel film based nitrite sensors employing substituted porphyrin dyes [J.Mater.Chem.11(2001)399], 2,3-diaminonaphthalene/cyclodextrin [J. Fluoresc.19(2009)119] and azobenzene dyes [Sens.ActuatorsB: 56(1999)15].

Reference also has to be made to disposible test strip sensor for nitrite which contain i)chemicals required for reaction and fixation of the dye formed with sulphanilamide, N-(1-naphthyl)ethylenediamine on Nafion [Anal.Bioanal.Chem.373(2002)289] ii) Greiss reagent [Anal. Lett.38(2005)1803] and iii)3-hydroxy-7,8-benzo-1,2,3,4-tetrahydroquinoline [J.Anal.Chem. 63(2008) 792].

Another reference may be made to formation of sol-gel glasses[Haruby and Webber, U.S. Pat. No. 5,272,240; Wang et al USP 2003/0147606A1; Lee et al U.S. Pat. No. 5,329,540], colorimetric sol-gel sensor for nitrite[Charych et al, U.S. Pat. No. 6,022,748] and nitrite test strip sensor[Kylor et al, USP 2005/0101841A9].

All of the above mentioned optical test strips designed for nitrite are based on diazo coupling reactions and are non selective, require long equilibration times, narrow calibration range and less sensitivity. On the other hand the method based on rhodamine 6G entrapped sol-gel film test strip offers virtually specific and sensitive determination of nitrite.

### OBJECTIVES OF THE INVENTION

The main objective of the present invention is to provide dye entrapped sol-gel film based test strip sensor for nitrite which obviates the drawbacks mentioned above.

Another object of the present invention is to provide a process for the preparation of rhodamine 6G entrapped sol-gel glass.

Yet another object of the present invention is to construct inexpensive and sturdy test strip sensors utilizing above sol-gel glass.

Still another object of the present invention is selective absorptiometric sensing of nitrite in presence of host of coexisting anions, cations and neutral salts which can tolerate high concentrations of acids.

### BRIEF DESCRIPTION OF ACCOMPANYING DRAWINGS

FIG. 1 summarizes chemical processes occurring during entrapment of rhodamine 6G in sol-gel glass film.

FIG. 2 represents schematic diagram depicting rhodamine 6G based solution and test strip sensors for visual detection and absorptiometric quantification.

FIG. 3 depicts the specificity of rhodamine 6G entrapped sol-gel film based test strip sensor for nitrite

FIG. 4 represents flow sheet for preparation of dye (rhodamine 6G) entrapped sol-gel glass films.

### SUMMARY OF THE INVENTION

Accordingly, present invention provides a dye entrapped sol-gel film based test strip sensor for nitrite in natural water comprising a substrate coated with a rhodamine 6G entrapped sol-gel film.

In an embodiment of the present invention, said substrate is a glass substrate. In yet another embodiment of the present invention, said strip exhibiting absorption and fluorescence maxima at 525 and 555 nm respectively.

In yet another embodiment of the present invention, said strip specifically senses nitrite in natural water the range of 0.04-0.12 ppm and had a limit of detection of 0.01 ppm in acidic medium.

In yet another embodiment of the present invention, said sensor specifically senses nitrite in 1-1.5N dilute sulphuric acid medium, washed preferably with solvent dried for a minimum of 10 minutes and by measuring spectrophotometrically at 525 nm.

In yet another embodiment of the present invention, solvent used is selected from the group consisting of methanol, ethanol or dichloromethane.

In yet another embodiment of the present invention, said sensor exhibiting stability upto 30 days.

In an embodiment, present invention provides a process for the preparation of dye entrapped sol-gel film based test strip sensor comprising the steps of:
i. sonicating a mixture of tetraethoxysilane(TEOS), water and ethanol in the ratio ranging between 1:2:3 to 1:5:5 for period in the range of 20 to 40 minutes to form sol;
ii. hydrolysing the sol as obtained in step (i) by drop wise addition of 0.05 to 0.1 M HCl under sonication;
iii. further sonicating the hydrolysed sol as obtained in step (ii) for 30 to 60 minutes after adding of 0.03 to 0.07% of rhodamine 6G;
iv. aging sonicated sol as obtained in step (iii) for period in the range of 15 to 21 hr to obtain dye entrapped sol-gel glass;
v. casting sol gel glass as obtained in step (iv) as sol gel films on glass substrates followed by drying for period in the range of 40 to 50° C. for period in the range of 9 to 15 hr to obtain test strip sensor.

### DETAILED DESCRIPTION OF THE INVENTION

Accordingly the present invention provides dye entrapped sol-gel film based test strip sensor for nitrite and a process thereof which comprises a process of casting of dye entrapped thin sol-gel film based test strips employing:
a) Preparation of rhodamine 6G entrapped sol-gel glass;
b) Casting of optically transparent test strip with above sol-gel glass;
c) Virtually specific sensing of nitrite.

### a) Preparation of Rhodamine 6G Entrapped Sol-Gel Glass

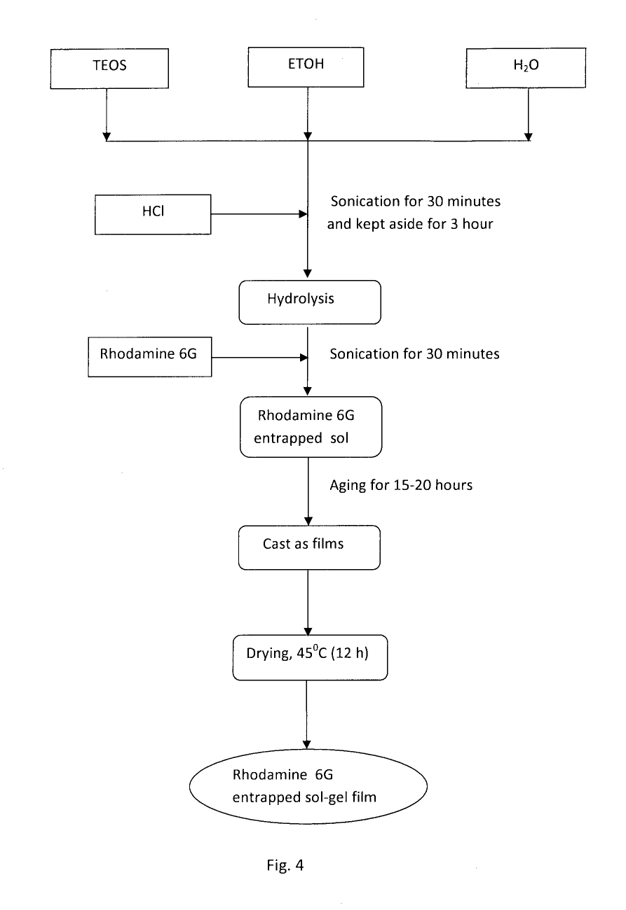

The experimental procedure for preparing dye entrapped sol-gel glass is shown in FIG. 4

There are three main steps in the preparation of dye entrapped sol-gel glass:

i. Identification of a Suitable Composition of Sol Conatining Tetraethoxysilane, Water and Ethanol

Different molar ratios of tetraethoxy silane (TEOS), ethanol and water during hydrolysis with 0.01 M HCl were screened for arriving at a suitable recipe which will give a transparent, reproducible test strip without leaching of the dye. Among several recipes tried, 1:3:5, 1:2:3 and 1:5:4 (TEOS:ethanol:water) were found to give test strip without leaching. However, the first one offered better reproducibility in nitrite quantification compared to other two and was preferred in subsequent studies.

ii. Hydrolysis of sol with Dropwise Addition of HCl Followed by Entrapment of Rhodamine 6G

0.20 ml of 0.01 M HCl was added drop wise and sonicated for 30 min and kept aside for 3 hours. The concentration of rhodamine 6G for entrapping in sol-gel film was varied from 0.001 to 0.01 g. Excess amount of dye resulted in inconsistent nitrite analytical signal, probably due to dimerization of the dye and too fewer amounts resulted in lesser sensitivity. An optimum of 0.005 g was chosen for further studies. The thorough mixing of rhodamine 6G with sol-gel recipe via sonication requires a minimum of 30 min and is unaffected on increasing to 60 min.

iii. Aging of the Sol to Form Sol-Gel Glass before Casting on Pretreated Glass Plates

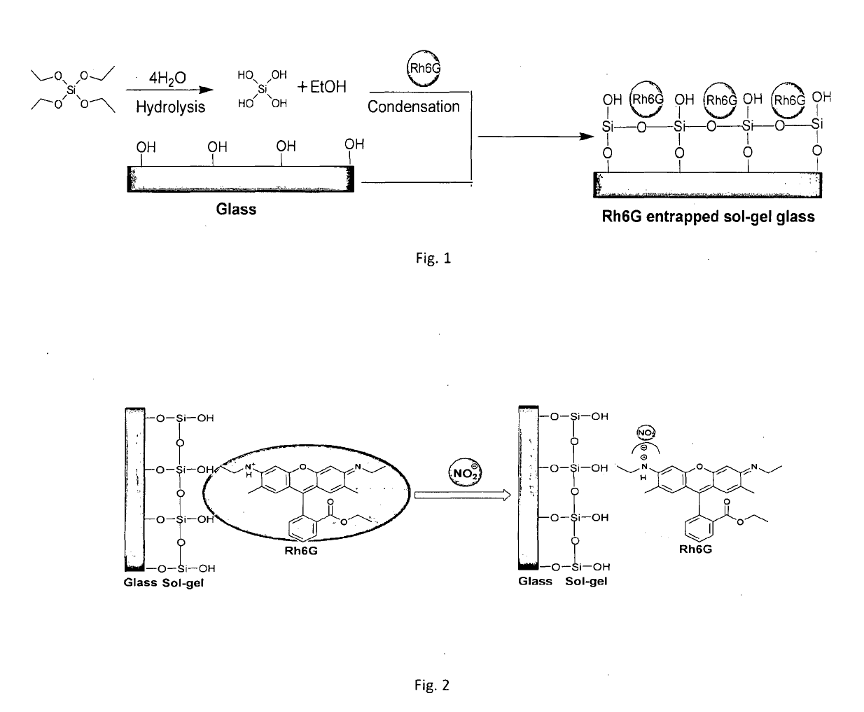

The ageing time of sol-gel recipe for entrapping rhodamine 6G, varied in steps of 5 h in the range of 5-100 h indicate an optimal ageing time of 15-20 h. The drying time and drying temperature for the formation of gel for the effective entrapment of rhodamine 6G was found to be 12 h and 45° C. respectively. FIG. 1 lists sequence of chemical steps occurring during sol-gel glass preparation.

b) Casting of Optically Transparent Test Strip with Rhodamine 6G Entrapped Sol-Gel Glass

Dye entrapped sol-gel film based test strips are constructed by manual casting of sol-gel glass on pretreated glass plates. The pretreatment of the glass plates were done with conc: HNO₃, distilled water and ethanol followed by drying. FIG. 2 depicts schematic diagram of rhodamine 6G based solution and test strip sensors for visual detection and absorptiometric quantification.

### c) Virtually Specific Sensing of Nitrite

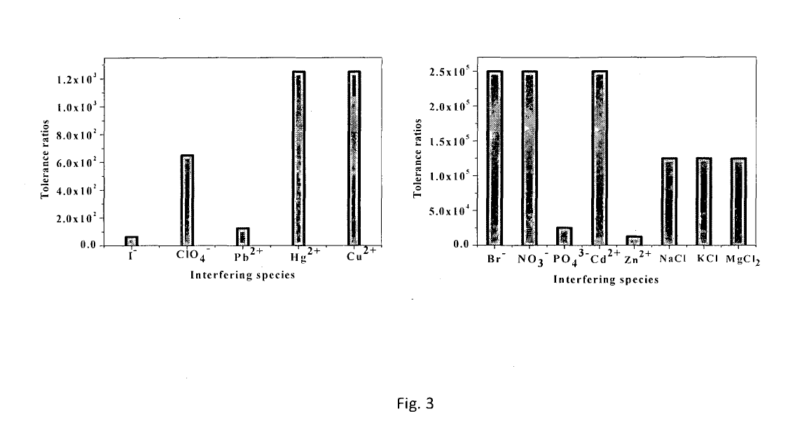

Test strips are exposed to nitrite (0.04-0.12ppm) solutions acidified with 1-1.5N H₂SO₄, washed preferably with methanol (ethanol and dichloromethane can also be used) and dried for a minimum of 10 minutes and absorbances were measured spectrophotometrically at λₘₐₓ of 525 nm. FIG. 3 highlights the specificity of the designed test strip sensor for nitrite over several coexisting species. The developed test strip finds application for determination of nitrite in natural waters (tap, well and sea waters) as determined by both direct and standard addition methods. The results obtained are shown below in table 1.

TABLE 1
Analysis of natural water samples

Nitrite concentration

| | | Direct method^{a} | Standard addition |
|---|---|---|---|
| Sl. No. | Sample | (ppm) | method (ppm) |
| 1 | Tap water | 0.092 ± 0.002 | 0.090 |
| 2 | Well Water | 0.043 ± 0.002 | 0.038 |
| 3 | Sea Water | 0.063 ± 0.002 | 0.060 |

^{a}Average of three determinations

Comparative sensing characteristics of nitrite test strip sensor with commercially marketted bio and electrochemical sensors are summarized in table 2.

TABLE 2

| | | | | Optical Sensor |
|---|---|---|---|---|
| | | | | Rhodamine |
| | | | | 6 G entrapped |
| | | | | Sol-gel based |
| | | Electrochemical Sensors | | Test strip of |
| | | | Orion | the present |
| Parameter | Biosensor | ELIT 8071 | 9346 | invention |
| Working | 0.05-36.80 | 0.5-46 | 0.02-100 | 0.04-0.12 |
| range | | | | |
| (ppm) | | | | |
| Limit of | 0.004 | — | 0.18 | 0.01 |
| detection | | | | |
| (ppm) | | | | |
| Response | 5 s | <10 s | — | 10 minutes |
| time | | | | |
| Sample | pH 6-8 | pH 4.5-8.0 | — | 1-1.5N H₂SO₄ |
| pH/H⁺ | | | | |
| Sample | 4° C. | 5-50° C. | 0-40° C. | RT |
| temperature | | | | |
| Stability | Less than | Storing in | Stable | Stable |
| | 1 week | refrigerator at | | |
| | due to | 5° C. is | | |
| | leaching | essential to | | |
| | of enzyme | avoid prolonged | | |
| | | preconditioning | | |
| Selectivity | Not | Fluoride, | Same as | Highly |
| | absolutely | Acetate Sulphate, | ELIT | selective |
| | selective | Nitrate | | |

### EXAMPLES

Following examples are given by way of illustration therefore should not be construed to limit the scope of the invention.

### Example 1

The rhodamine 6G entrapped sol-gel film based test strip was prepared by sonicating for 30 minutes a mixture of 0.420 ml of tetraethoxysilane, 0.55m1 of ethanol and 10 ml of water to form sol and then hydrolysing by dropwise addition of 0.1M HCl (0.2 ml ) under sonication. This gel is further sonicated for 45 minutes after addition of 0.005 g of rhodamine 6G, aged for 18 h, cast as films and dried at 45° C. for 12 h.

### Example 2

The rhodamine 6G entrapped sol-gel film based test strip was prepared by sonicating for 30 minutes a mixture of 0.630 ml of tetraethoxysilane, 0.490 ml of ethanol and 10 ml of water to form sol and then hydrolysing by dropwise addition of 0.1M HCl (0.2 ml ) under sonication.This is further sonicated for 45 minutes after addition of 0.005 g of rhodamine 6G, aged for, 18 h cast as films and dried at 45° C. for 12 h.

### Example 3

The rhodamine 6G entrapped sol-gel film based test strip was prepared by sonicating a mixture of 0.250 ml of tetraethoxysilane, 0.260 ml of ethanol and 10 ml of water for 30 minutes to form a sol and then hydrolysed by dropwise addition of 0.1M HCl (0.2 ml ) under sonication. This gel is further sonicated for 45 minutes after addition of 0.005 g of rhodamine 6G, aged for 18 h cast as films and dried at 45° C. for 12 h.

### Example 4

The sol-gel recipe of Example 1 is prepared with 0.003, 0.004, 0.006 and 0.007 g of rhodamine 6G.The sol-gel recipe of Example 1 is prepared by sonicating for 30 and 60 min after addition of rhodamine 6G.The sol-gel recipe of Example 1 is prepared by aging for 15 to 20 hours before casting as films. The dye entrapped sol-gel test strip is prepared by drying at 45° C. for 10 to 15 hours.

### Example 5

The stability of rhodamine 6G entrapped sal-gel films were tested over 0, 5, 10, 20 and 30 days and found to be good.

| | No. of days | | | | |
|---|---|---|---|---|---|
| | 0 | 5 | 10 | 20 | 30 |
| Absorbance | 0.718 | 0.701 | 0.700 | 0.698 | 0.698 |
| at 525 nm | | | | | |

### Calibration Graph and Virtually Specific Sensing of Nitrite

### Example 6

The rhodamine 6G entrapped sol-gel film based test strip senses 0.04-0.12 ppm of nitrite. The calibration data of test strip is given below.

| | Concentration of | | |
|---|---|---|---|
| | nitrite (ppm) | | |
| | 0.04 | 0.08 | 0.12 |
| ΔA (Aₛₐₘₚₗₑ − A_{blank}) | 0.041 | 0.067 | 0.095 |

The selectivity of rhodamine 6G entrapped sol-gel film for nitrite determination over several anionic, cationic and neutral electrolyte species that are known to coexist in natural waters was ascertained. The results as obtained in terms of tolerance ratios during determination of 40 ppb of nitrite are compiled in FIG. 3. The tolerance limit was defined as the concentration of added species causing less than ±5 relative error on the determination of 40 ppb of nitrite. Anions (10⁵ fold amounts of Br⁻, NO₃⁻, PO₄³⁻, 63 fold of iodide and 650 fold of CIO₄⁻), cations (10²-10⁵ fold amounts of Pb²⁺, Cu²⁺, Hg²⁺and Cd²⁺) and neutral salts (10⁵ foldamounts of NaCl, KCl and MgCl₂) do not interfere.

### Method of Utilization of Test Strips Example 7

The stable rhodamine 6G entrapped sol-gel film based test strips prepared by the combination of steps involving sol preparation, hydrolysis, dye entrapment, ageing, casting as films and drying as per the above examples can be used for virtually specific sensing of nitrite in presence of host of coexisting anions and cations as mentioned in the detailed description of the invention. The comparative account of present test strip based sensor with commercially marketted and literature reported nitrite bio-chemical and electrochemical sensors is given in table 2 which showed superiority.

The sol-gel based test strip provides the following characteristics.
i. Its dimension is 5 cm×1 cm(length& breadth)
ii. 0.2 ml of sol is used to cast a test strip.

1 iii. It shows a absorption and fluorescence maxima at 525 and 555 nm respectively.

### ADVANTAGES OF THE PRESENT INVENTION

1. Developed sol-gel based test strip sensor for nitrite.
2. The invented test strip based nitrite analysis is rapid, simple, inexpensive and highly selective.
3. The invented sol-gel based test strip sensor can tolerate high acidities.
4. The invented sol-gel based test strip sensor has better shelf life. Hence, it is economically viable and environmental friendly.
5. The developed sol-gel based test strip sensor can be used for the virtually specific absorptiometric sensing of nitrite in natural waters.

We claim:

1. Dye entrapped sol-gel film based test strip sensor for nitrite in natural water comprising a substrate coated with a rhodamine 6G entrapped sol-gel film.

2. The test strip sensor as claimed in claim 1, wherein said substrate is a glass substrate.

3. The test strip sensor as claimed in claim 1, wherein said strip exhibiting absorption and fluorescence maxima at 525 and 555 nm respectively.

4. The test strip sensor as claimed in claim 1, wherein said strip specifically senses nitrite in natural water the range of 0.04-0.12 ppm and had a limit of detection of 0.01ppm in acidic medium.

5. A sensor as claimed in claim 1, wherein said sensor specifically senses nitrite in 1-1.5N dilute sulphuric acid medium, washed preferably with solvent dried for a minimum of 10 minutes and by measuring spectrophotometrically at 525nm.

6. A sensor as claimed in claim 3, wherein solvent used is selected from the group consisting of methanol, ethanol or dichloromethane.

7. A sensor as claimed in claim 1, wherein said sensor exhibiting stability upto 30 days.

8. A process for the preparation of dye entrapped sol-gel film based test strip sensor as claimed in claim 1, wherein said process comprising the steps of:
i. sonicating a mixture of tetraethoxysilane(TEOS), water and ethanol in the ratio ranging between 1:2:3 to 1:5:5 for period in the range of 20 to 40 minutes to form sol;
ii. hydrolysing the sol as obtained in step (i) by drop wise addition of 0.05 to 0.1 M HCl under sonication;
iii. further sonicating the hydrolysed sol as obtained in step (ii) for 30 to 60 minutes after adding of 0.03 to 0.07% of rhodamine 6G;
iv. aging sonicated sol as obtained in step (iii) for period in the range of 15 to 21 hr to obtain dye entrapped sol-gel glass;
v. casting sol gel glass as obtained in step (iv) as sol gel films on glass substrates followed by drying for period in the range of 40 to 50° C. for period in the range of 9 to 15 hr to obtain test strip sensor.
